(19) United States
(12) Patent Application Publication
(10) Pub. No.: US 2015/0005587 A1
(43) Pub. Date: Jan. 1, 2015

(54) GOGGLES FOR EMERGENCY DIAGNOSIS OF BALANCE DISORDERS

(71) Applicants: Yinhong Qu, Tianjin (CN); Ian Purcell, San Diego, CA (US)

(72) Inventors: Yinhong Qu, Tianjin (CN); Ian Purcell, San Diego, CA (US)

(21) Appl. No.: 13/929,572

(22) Filed: Jun. 27, 2013

(51) Int. Cl. A61B 5/00; A61B 3/00; A61B 3/14; A61B 5/11; A61B 3/113

(52) U.S. Cl. CPC A61B 5/4023; A61B 5/1112; A61B 3/113; A61B 3/14; A61B 5/6803; A61B 3/0041; A61B 5/7405
USPC 600/301

(57) ABSTRACT

A system for use by a clinician to perform an emergency diagnosis of a patient for a balance disorder includes a position sensor and an optical sensor that are mounted on a base member. The base member is configured to be fixedly held over the eyes of the patient during a predetermined diagnostic maneuver of the head of the patient. A processor having electronic memory is included which receives outputs from the optical sensor and the position sensor. A set of instructions constituting an eyetracker computer program is processed by the computer. Quantified eye movement data and a video stream signal can be displayed on a visual display that is affixed to the base member for observation by the clinician and recorded in computer readable memory.

### FIELD OF THE INVENTION

The present invention pertains generally to the diagnosis of balance disorders. More particularly, the present invention pertains to systems and methods for diagnosing positional vertigo by observing a patient's eyes during a diagnostic maneuvering of the patient's head. The present invention is particularly, but not exclusively, useful as a system that can be used by a clinician to perform an emergency diagnosis of a patient for a balance disorder.

### BACKGROUND OF THE INVENTION

Vertigo is a common medical condition in which a person temporarily experiences dizziness, a perceived spinning motion, and/or a general feeling of imbalance that can cause difficulty in standing or walking. Patients often seek medical attention after experiencing vertigo, with many of these patients choosing to find immediate treatment in an emergency room setting.

Although vertigo symptoms can result from several different causes, including certain head injuries. In any event, a large percentage of patients that have experienced vertigo are diagnosed with Benign Paroxysmal Positional Vertigo (BPPV). In simple terms, BPPV is an inner ear condition which causes vertigo to occur when the position of a person's head changes in a specific way. For example, BPPV associated vertigo may occur when the person's head is tilted or when a person rolls over in bed.

In more anatomical terms, BPPV can be caused when calcium crystals that are normally present in the inner ear are disturbed and assume new positions within the inner ear. Once the calcium crystals have been somehow repositioned, certain movements of the person's head can cause fluid in the inner ear to undergo an abnormal displacement, which in turn, results in vertigo.

During vertigo, involuntary eye movement called Nystagmus can occur. Importantly, the involuntary eye movement is observable and can be detected during a diagnostic maneuver. For example, the so-called Dix-Hallpike test can be used to identify the cause of a patient's vertigo. Specifically, the Dix-Hallpike test can be used to determine whether vertigo is caused by an abnormality in the patient's brain (central vertigo) or an abnormality such as BPPV in the inner ear (peripheral vertigo). During the test, the affected patient's eyes are monitored, together or individually, for involuntary movement(s). Specifically, this is done while the patient's head is carefully moved through a predetermined sequence of head orientations. When the test is properly performed and the results are properly evaluated, the test can be used to determine whether the vertigo is caused by an inner ear condition, such as BPPV, and it can provide an indication as to which ear (right or left) is causing the vertigo.

In an emergency room (ER) setting, it may be very helpful, if possible, to diagnose vertigo. It can happen, however, that there may be no trained clinician on-staff at the time a patient is admitted to the ER. In such situations an operable system for assisting a clinician during vertigo testing, and for creating a record of the test, which can be used for subsequent review and evaluation, may be particularly helpful.

In light of the above, it is an object of the present invention to provide systems and methods for assisting a clinician in an emergency setting to diagnose positional vertigo. It is another object of the present invention to provide systems and methods which record a patient's eye movement during a diagnostic maneuvering of the patient's head. Still another object of the present invention is to provide a system for tracking and recording both horizontal and vertical coordinates of involuntary eye movements which occur during a diagnostic maneuvering of the patient's head. Yet another object of the present invention is to provide goggles for emergency diagnosis of balance disorders and corresponding methods of use which are easy to use, relatively simple to implement, and comparatively cost effective.

### SUMMARY OF THE INVENTION

In accordance with the present invention, a system for use by a clinician to perform an emergency diagnosis of a patient for a balance disorder includes a position sensor and an optical sensor that are both mounted on a base member. For the system, the base member is configured to be fixedly held over the eyes of the patient during a predetermined maneuver of the head of the patient. For example, the base member can be in the form of a pair of goggles that are worn by the patient and can include a strap for securing the goggles to the patient's head. Maneuvers can include, but are not limited to, the well-known Dix-Hallpike maneuver, Supine Positional Testing, and an Epley maneuver.

With the base member secured to the patient, the position sensor can output information (i.e. a data signal) regarding the patient's head orientation at preselected locations during the maneuver. For example, the position sensor can include gyroscopes, GPS receivers or other suitable position sensors known in the pertinent art. In addition, the optical sensor is oriented on the base member such that when the base member is secured on the patient's head, a series of images (i.e. a video image) of at least one of the patient's eyes is acquired by the optical sensor. The optical sensor then outputs an electrical signal corresponding to the video image.

For the system, a computer having electronic memory is included which receives the outputs from the optical sensor and the position sensor. A set of instructions constituting an eyetracker computer program is processed by the computer. The computer and eyetracker program function to identify the position of the eye (e.g. relative to the head) in the video image signal and output the coordinates (e.g. horizontal and vertical coordinates) of the eye as a function of time. It will also be appreciated that combinations of horizontal and vertical movements will be manifested as torsional movements. Typically, these coordinates are measured relative to a reference eye position. In more detail, the eyetracker program is performed to identify and quantify involuntary eye movements for each of the selected head orientations in the maneuver. The quantified eye movement data (horizontal, vertical, and torsional), and video stream signal can then be recorded in computer readable memory.

Also for the system of the present invention, a visual display is affixed to the base member and oriented to allow a clinician to observe the display during a maneuver. In addition, the system can include a speaker. The visual display and speaker are connected to, and receive signal outputs from, the computer. With this cooperative arrangement, the visual display can present a magnified view of one or both of the patient's eyes and can simultaneously include quantified eye movement data, in real time, for example in graphical and/or tabular form. In addition, the speaker can provide audio prompts for guiding the clinician when orienting the head of the patient at each of the preselected locations in accordance with the maneuver.

In an operation of the present invention, the system is donned by the patient and activated. Once activated, position data corresponding to the selected maneuver can be input into the computer. Next, the clinician manipulates the patient's head into the first head orientation in accordance with the maneuver.

When the proper head orientation is achieved by the clinician, it is verified. A signal is then sent from the computer to the speaker to generate an audio prompt for reassuring the clinician that the maneuver is being performed correctly. Once proper head orientation is verified, and stabilized, the patient's head is then held at the first selected orientation for a preselected period of time. During this time, a magnified view of the patient's eye(s) and the quantified eye movement data are displayed for clinician observation. The displayed video and data can also be stored in memory, such as an archive unit, for subsequent review.

Next, the clinician manipulates the patient's head toward another selected head orientation until the audio prompt sounds, indicating that the correct orientation has been achieved. The patient's head orientation is then verified, and the head is held at the selected orientation for a preselected period of time. Once again, a magnified view of the patient's eye(s) and the quantified eye movement data are displayed for clinician observation and recorded. This process in then repeated until all of the orientations in the maneuver are completed. The data can then be evaluated by the clinician to diagnose the cause of the patient's vertigo.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a front view of a patient wearing a system for assisting a clinician in performing an emergency diagnosis for a balance disorder in accordance with the present invention;

FIG. 2 is a schematic diagram of the operational components for the system shown in FIG. 1; and

FIG. 3 is a flowchart diagram illustrating a procedure for diagnosing a balance disorder using the system 10 shown in FIG. 1.

### DESCRIPTION OF THE PREFERRED EMBODIMENTS

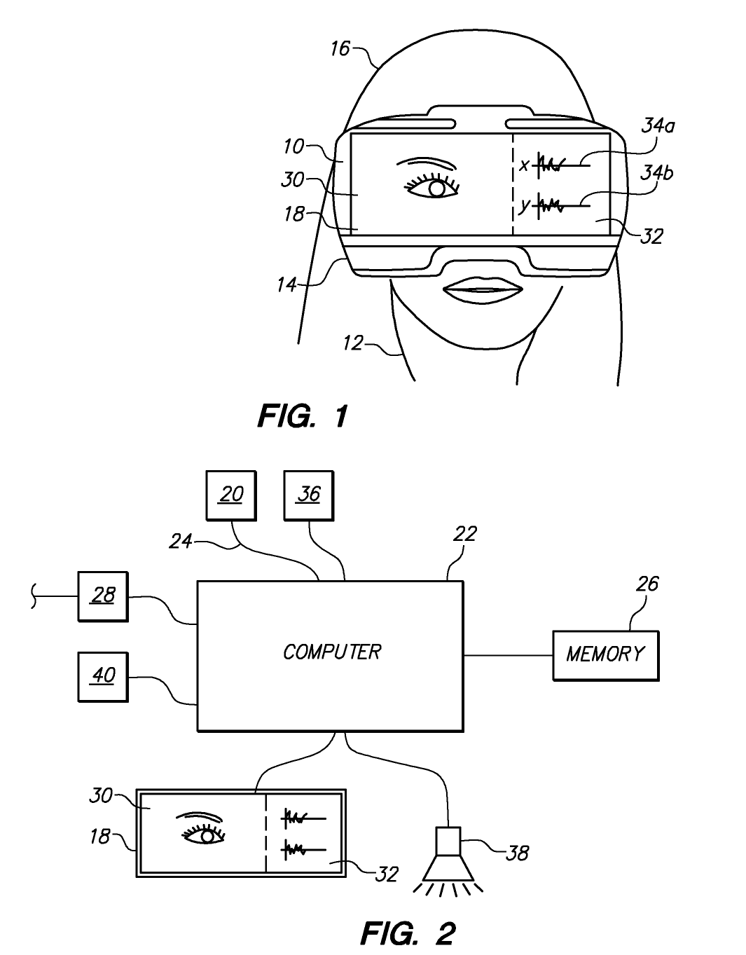

With initial reference to FIG. 1, a system 10 for use by a clinician to perform an emergency diagnosis of a patient 12 for a balance disorder is shown. As shown, the system 10 includes a base member in the form of a set of goggles 14 that are fixedly held over the eyes of the patient 12 during a predetermined maneuver of the head 16 of the patient 12. FIG. 1 also shows that a visual display 18, which may be, for example, an LCD or LED screen, is mounted on the goggles 14 and is oriented to allow a clinician (not shown) to observe the display 18 during a maneuver (described in greater detail below).

FIG. 2 shows other components of the system 10 that can be mounted on the goggles 14 shown in FIG. 1. These components include an optical sensor 20 that is positioned on a surface of the goggles 14 opposite the display 18 such that the optical sensor 20 can image one or both of the eyes of the patient 12 when the goggles 14 are secured on the patient's head 16. For example, the optical sensor 20 can be a CCD or CMOS sensor similar to the optical sensors used on mobile phone cameras or any other type of optical sensor known in the pertinent art that can achieve the functionality described herein. A light can be included with the optical sensor 20 to facilitate imaging.

Continuing with reference to FIG. 2, the optical sensor 20 outputs an electrical signal encoding video image data of the patient's eye or eyes which is received by a computer 22 via transmission line 24. As shown, the computer 22 is connected to electronic memory 26 allowing the computer 22 to read from and/or write instructions and data to memory 26. In addition, a communications port 28, such as a Universal Serial Bus (USB) port, can be provided, allowing the computer to transmit and receive data and/or computer instructions (i.e. software code) to and from an external source (not shown), such as an external computer, computer network or external memory drive. The system 10 can also include wireless communication capability (not shown) such as Bluetooth, Wi-Fi or any other wireless protocol known in the pertinent art.

The image data from the optical sensor 20 is used with a set of computer readable instructions constituting an eyetracker computer program. Any program known in the pertinent art for tracking the movement of an eye can be used. Operational control instructions for calibration, user interaction and so forth can also be provided. The eyetracker instructions, which may reside in memory 26, are processed by the computer 22 to identify the position of the eye (e.g. relative to the head 16) in the video image signal. The computer 22 then outputs the coordinates (e.g. horizontal and vertical coordinates and their combinations) of the eye as a function of time, relative to a reference eye position. For example, the reference eye position can be acquired as an initial eye position when the patient's head 16 is oriented at one of the preselected locations during the maneuver, or the reference eye position can be acquired during an eyetracker calibration step (see below). The eyetracker program is performed to identify and quantify involuntary eye movements for each of the selected head orientations in the maneuver. For the system 10 shown in FIG. 1, the quantified eye movement data and video stream signal can be recorded in computer readable memory 26.

FIGS. 1 and 2 show that the visual display 18 can include a display portion 30 that presents a magnified view of one or both of the patient's eyes and can simultaneously present a display portion 32 that includes quantified eye movement data, in real time, for example in graphical form, as shown, and/or tabular form (not shown). For the graphical presentation shown, graph 34a presents the horizontal distance of the eye from the reference eye position (ordinate) as a function of time (abscissa) and graph 34b presents the vertical distance of the eye from the reference eye position (ordinate) as a function of time (abscissa). Other presentation formats may be used to display the quantified eye movement data.

In an exemplary embodiment, about two-thirds of the visual display 18 can include a magnified view of one or both of the patient's eyes and about one-third of the visual display 18 can include quantified eye movement data. Magnification can assist the clinician in viewing and evaluating involuntary eye movements and can be accomplished optically and/or digitally. For example, to do this, the optical sensor 20 can include one or more magnifying lens(es). Alternatively, a magnified image can be produced by processing the image data to produce an enlarged view using a digital zoom technique.

FIG. 2 also shows a position sensor 36 and speaker 38 can be included in the system 10 shown in FIG. 1 and operably coupled with the computer 22. The position sensor 36 can output information (i.e. a data signal) regarding the patient's head orientation and/or position at preselected locations during the maneuver. For example, the position sensor 36 can include one or more gyroscopes and/or accelerometers generating position and/or inclination information relative to a reference head position and/or inclination. Alternatively, or in addition to the gyroscopes and/or accelerometers, the position sensor 36 can include a GPS receiver operably connected to the computer 22 for receiving and processing GPS signals to determine a position and/or orientation of the patient's head 16. With this cooperative interaction of structural components, the speaker 38 can provide audio (e.g. voice) prompts for guiding the clinician (not shown) when orienting the head 16 of the patient 12 at each of the preselected locations in accordance with the maneuver.

FIG. 2 also shows that a user input terminal 40 can be operably coupled with the computer 22 to allow a user, such as the clinician, to activate, calibrate, reset, program and/or perform other interactive operations with the system 10 shown in FIG. 1. For example, the visual display 18 can be a touchscreen allowing the user (clinician) to interact with the system 10. For this case, a power switch (not shown) is typically also included and mounted on the goggles 14. Alternatively, or in addition to a touch screen, the system 10 shown in FIG. 1 can include one or more buttons, dials, switches and/or indicator lights to allow the user to interact with the device.

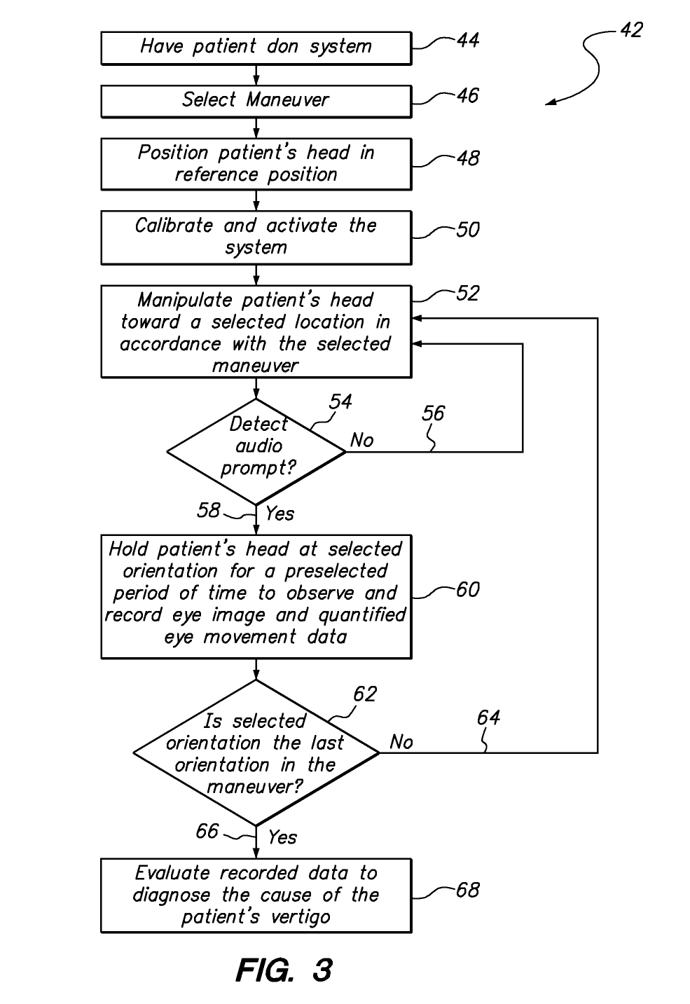

FIG. 3 illustrates an operation 42 of the system 10 shown in FIG. 1 and described above. As shown, the operation 42 can begin by affixing the system 10 on the patient 12 (Box 44) as shown in FIG. 1. Next, for a system which is set up to include multiple maneuvers, the clinician can select the applicable maneuver (Box 46). As envisioned for the present invention, this capability includes, at least, a selection between well-known diagnostic tests such as a Dix-Hallpike maneuver, an Epley maneuver or a sequence for Supine Positional Testing. In the event, the user input 40 shown in FIG. 2 can be used to interact with the system 10 and select the appropriate maneuver. As an example, a user driven menu screen can be presented on the display 18 by the computer 22 as the computer 22 processes operational control instructions.

For purposes of discussion, a typical Dix-Hallpike Maneuver requires head movements through a sequence of seven separate head orientations. In order, these orientations are:

1. patient sits with head centered;

2. patient sits with head 45 degrees to the right;

3. patient lays back with head 45 degrees to the right and held in approximately 20 degrees of extension;

4. patient sits back up with head centered;

5. patient sits with head 45 degrees to the left;

6. patient lays back with head 45 degrees to the left and held in approximately 20 degrees of extension; and

7. patient sits back up with head centered.

For the system 10, position information (relative or absolute) for each location where observations will be conducted can be stored in memory 26 for each maneuver that is available to the clinician. This position information is then used in a comparison subroutine to provide audio (e.g. voice) prompts during the maneuver, as detailed further below.

Next, Box 48 indicates that the patient's head is placed in an initial position, which may also, in some cases, be used as a reference position for calculating the positions of each location where observations will be conducted during the maneuver. Box 50 indicates that the system 10 can be activated and, in some cases, calibrated. Specifically, a calibration can be performed to establish a reference eye position for use in the eyetracker program. Further, proper positioning in the reference position can be verified.

Once activated and calibrated, the clinician manipulates the patient's head 16 toward the first selected head orientation as indicated by Box 52. As the head is manipulated into position, the computer 22 compares the actual position from the position sensor output with the pre-programmed position (which may be a range of acceptable positions) to determine when the head 16 is in the prescribed position in accordance with the maneuver. This is then verified. As FIG. 3 shows, the clinician can listen for an audio prompt and/or voice command (Box 54) while manipulating the head 16. If there is no prompt (Box 56), further manipulation (Box 52) is required until the audio prompt is sounded (Box 58). Again, verification of the proper position is made.

Once the proper head orientation is achieved by the clinician, the patient's head 16 is then stabilized and held at the first selected orientation for a preselected period of time (Box 60). During this time, while the head is stabilized and verified, a magnified view of the patient's eye(s) and the quantified eye movement data are displayed for clinician observation and can be recorded.

Next, after the preselected period of time, and if the current orientation is not the last orientation of the selected maneuver (Boxes 62 and 64), the clinician manipulates the patient's head 16 toward another selected head orientation (under voice command) until the audio prompt sounds, indicating that the correct orientation has been achieved (Box 52). On the other hand, if the current orientation is the last orientation of the selected maneuver (Boxes 62 and 66), the data can then be evaluated by the clinician to diagnose the cause of the patient's vertigo (Box 68).

While the particular goggles for emergency diagnosis of balance disorders as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for use by a clinician to perform an emergency diagnosis of a patient for a balance disorder which comprises:
a base member, wherein the base member is fixedly held over the eyes of the patient during a predetermined maneuver of the head of the patient;
a position sensor mounted on the base member to provide information for identifying an orientation of the head of the patient at preselected locations during the maneuver;
an optical sensor mounted on the base member for respectively viewing the eyes of the patient at a sequence of preselected locations during the maneuver;
an eyetracker mounted on the base member for recording quantified eye movement data during the maneuver, wherein the quantified eye movement data is collected at each preselected location, for presentation pertinent to respective orthogonal directions;
a visual display affixed to the base member for observation by the clinician during the maneuver, wherein the visual display is connected to the optical sensor to present an observable visualization of eye movements during the maneuver in real time, and is connected to the eyetracker to simultaneously present corresponding quantified eye movement data; and
an audio prompt for guiding the clinician when orienting the head of the patient at each of the preselected locations in accordance with the maneuver.

2. A system as recited in claim 1 wherein the maneuver is selected from the group of maneuvers consisting of a Dix-Hallpike maneuver, an Epley maneuver and a Supine Positional Testing Sequence.

3. A system as recited in claim 2 wherein the maneuver includes supine positional testing.

4. A system as recited in claim 1 wherein the base member is a set of goggles.

5. A system as recited in claim 1 wherein the position sensor incorporates GPS technology.

6. A system as recited in claim 1 wherein eye movements are magnified for presentation on the visual display while the head of the patient is stabilized.

7. A system as recited in claim 1 wherein the orthogonal directions are horizontal and vertical, and a combination of these directions can be observed as clockwise and counterclockwise torsional eye movements.

8. A system as recited in claim 1 further comprising an archive unit for storing a visual record of the eye movements obtained from the optical sensor, and the corresponding quantitative eye movement data, to include moving and stationary head position data.

9. A system for use by a clinician to perform an emergency diagnosis of a patient for a balance disorder which comprises:
a base member, wherein the base member is fixedly held over the eyes of the patient during a predetermined maneuver of the head of the patient;
an optical sensor mounted on the base member and oriented to view at least one eye of the patient and create image data of the eye;
a computer for receiving said image data and for processing a set of computer instructions constituting an eyetracker computer program and recording quantified eye movement data during the maneuver; and
a visual display affixed to the base member for observation by the clinician during the maneuver, wherein the visual display is connected to the optical sensor to present an observable visualization of eye movements during the maneuver in real time, and is connected to the processor to simultaneously present corresponding quantified eye movement data.

10. A system as recited in claim 9 further comprising:
a position sensor mounted on the base member and providing an output to the computer and wherein the computer processes the position sensor output to confirm an orientation of the head of the patient at preselected locations during the maneuver; and
a speaker connected to the computer to produce an audio prompt for guiding the clinician when orienting the head of the patient at each of the preselected locations in accordance with the maneuver.

11. A system as recited in claim 9 wherein the maneuver is selected from the group of maneuvers consisting of a Dix-Hallpike maneuver and an Epley maneuver.

12. A system as recited in claim 9 wherein the base member is a set of goggles.

13. A system as recited in claim 9 wherein the position sensor incorporates GPS technology.

14. A system as recited in claim 9 wherein eye movements are magnified for presentation on the visual display.

15. A system as recited in claim 9 wherein the quantified eye movement data is collected at each preselected location for presentation pertinent to respective orthogonal directions relative to a reference eye position.

16. A method for performing an emergency diagnosis of a patient for a balance disorder by a clinician, the method comprising the steps of:
mounting an optical sensor and a visual display on a base member;
holding the base member over the eyes of the patient with the visual display oriented for observation by the clinician and with the optical sensor oriented to view at least one eye of the patient to create image data of the eye;
maneuvering the head of the patient into a plurality of verified head orientations;
stabilizing the head of the patient at each head orientation;
receiving image data from the optical sensor at a computer during the stabilizing step;
processing the image data and a set of eyetracker computer program instructions to generate quantified eye movement data during the maneuver; and
displaying the quantified eye movement data on the visual display during the maneuver.

17. A method as recited in claim 16 further comprising the step of:
presenting a magnified image of at least one eye of the patient, in real time, on the visual display during the maneuver.

18. A method as recited in claim 16 wherein the maneuver is selected from the group of maneuvers consisting of a Dix-Hallpike maneuver and an Epley maneuver.

19. A method as recited in claim 18 wherein the maneuver includes Supine Positional Testing.

20. A method as recited in claim 16 further comprising the steps of:
mounting a position sensor and a speaker on the base member;
receiving a position sensor output at the computer during the maneuver;
processing the position sensor output at the computer to confirm an orientation of the head of the patient at preselected locations during the maneuver; and
sending a signal from the computer to the speaker to produce an audio prompt for guiding the clinician when orienting the head of the patient at each of the preselected locations in accordance with the maneuver.
